(19) United States
(12) Patent Application Publication
(10) Pub. No.: US 2003/0171954 A1
(43) Pub. Date: Sep. 11, 2003

(54) Method of managing the provision of healthcare and system for effecting same

(75) Inventors: Joel F. Guerin, Versailles, KY (US); Ricky J. Fontaine, Lexington, KY (US); David P. Morgan, Midlothian, VA (US); Phillip J. O'Neill, Bennington, VT (US)

Correspondence Address: MICHAEL BEST & FRIEDRICH, LLP, 100 E WISCONSIN AVENUE, MILWAUKEE, WI, 53202, US

(73) Assignee: Tempur World, Inc., Lexington, KY

(21) Appl. No.: 10/356,376

(22) Filed: Jan. 31, 2003

Related U.S. Application Data
(60) Provisional application No. 60/353,998, filed on Feb. 1, 2002.

(51) Int. Cl. G06F 17/60; A61N 1/10; A61N 1/08; A61N 1/18; A61N 1/20; A61N 1/32; A61B 5/00

(52) U.S. Cl. 705/2; 600/300; 607/41; 607/70; 604/289

(57) ABSTRACT

A healthcare management program designed to reduce the risk of having adverse outcomes, and the costs of liability associated with those adverse outcomes, during the provision of healthcare. The management program is provided by a healthcare vendor to a healthcare provider for a fixed fee. The management program includes a plan of care that defines protocols to utilize during the provision of care to customers, documentation tools that provide evidence of the treatment and progress of each customer, and indemnity instruments which distribute the risks associated with the provision of care between the healthcare provider and the healthcare vendor.

### BACKGROUND OF THE INVENTION

The present invention relates to managing the provision of healthcare and, more particularly, to an outcomes-focused model for managing the provision of healthcare.

The provision of healthcare to individuals has been greatly effected by a number of factors. These factors include, among other things, changes in the reimbursement models utilized to compensate healthcare providers, changes in regulations related to the standard of care an individual can expect (i.e., patient's rights), an increase in litigious activity related to the provision of healthcare, an increase in the liability healthcare providers must assume, and an increase in the costs associated with insurability of healthcare providers.

For many years, the financial relationship between healthcare providers and individuals or customers (e.g., patient, resident, and the like) was largely based on the concept of “fee for service.” A fee paid to a healthcare provider by an individual was commonly negotiated between the healthcare provider and the individual, often on an ability to pay basis. Because the fee came directly from the individual, the individual was generally rather price sensitive. This price sensitivity helped to keep the costs associated with the provision of healthcare in-check.

When an increasing number of employers began to offer health care insurance packages to employees, the relationship between the healthcare provider and the individual began to change. Instead of the fee coming directly from the individual, the insurance provider was now generally paying the fee with the individual and/or the individual's employer paying set premiums to the insurance provider for such services. This new relationship resulted in a decrease in the amount of price sensitivity the individual exhibited. Accordingly, the costs associated with the provision of healthcare began to increase significantly as individuals came to expect healthcare as a right rather than as a privilege. With the increase in costs, insurance providers began to increase the costs associated with insurability.

Some organizations looked at evolving healthcare into a more comprehensive relationship to combat the startling increase in costs. One example of such a relationship is known as a Health Maintenance Organization (“HMO”). The HMO relationship, and other similar type relationships, essentially contract healthcare providers to provide healthcare products and/or services to subscribers at predetermined rates. The HMO type relationships proved to be cost effective for a large percentage of the population and evolved into other managed relationships which also gained in popularity as a result of each respective relationship's ability to manage costs and keep premiums relatively low.

Elements of these managed relationships were utilized in developing a relationship which became known as a Prospective Payment System (“PPS”). The PPS relationship establishes a standard fee, based on the average cost of caring for individuals found within similar diagnostic related groups, which is paid to a healthcare institution (e.g., a hospital) upon admission of a subscriber (e.g., a Medicare beneficiary). The PPS relationship was eventually extended to include long-term healthcare institutions (e.g., Medicare skilled nursing facilities). For a nursing facility, a case-mix fee is paid to the nursing facility based on the average cost of providing services to individuals within identified resource utilization groups (“RUGs”). Introduction of the PPS relationship to nursing facilities resulted in a shift from per-diem payments based on an individual historical cost report plus reimbursement for ancillary (i.e., non-routine) services (i.e., cost-plus), to a variable, all-inclusive case-mix payment based on acuity (i.e., fixed fee or cost).

Under fixed fee models, such as the PPS, a healthcare provider must provide the products and services necessary to achieve an acceptable outcome based on payment received at admission of the individual. The healthcare provider must continue to provide healthcare to the individual until the individual is stabilized in accordance with the admission criteria. The healthcare provider is rewarded for achieving a good outcome in the most timely and cost-efficient way, and is similarly penalized for poor or protracted outcomes. The funding the healthcare provider receives is intended to cover the cost of normal care. The healthcare provider keeps any excess in payment and pays for any shortfalls in payment.

While each of the fixed fee payment models has had a positive impact on containing the rate of healthcare cost growth, some believe that the fixed fee payment models have had a negative impact on the quality of care being delivered at some healthcare institutions. In response to these concerns, regulations related to the care of individuals have been enhanced. Included in these regulations are clearly identified “standards of care” which healthcare providers are required to follow. It is these standards of care which have acted as a catalyst to healthcare related litigation.

In situations where an individual may have an adverse clinical outcome, an institutional history of such outcomes, regardless of the trivial nature, has allowed for the establishment of a history of neglect compounding the seriousness of adverse clinical outcomes that arguably might not have been the healthcare institution's fault. Success in linking these issues has resulted in a series of increasingly high damage awards by juries in many states.

The magnitude of these settlements/judgements has caused extreme increases in medical malpractice and professional liability insurance premiums. In certain states, insurance, if it is even available, has become so prohibitively expensive that many healthcare providers have little choice other than to self-insure or go without coverage. However, financial institutions often require proof of insurance when healthcare providers are attempting to obtain financing.

In an environment where healthcare providers are pressured to reduce the costs associated with the provision of healthcare while still maintaining high standards of care, healthcare vendors have continued to receive payment based on non-fixed fee models. Although healthcare vendors have altered their business practices to a certain extent, healthcare vendors are still not willing to offer goods and/or services on a fixed fee basis.

### SUMMARY OF THE INVENTION

Accordingly, the invention provides a method of managing the provision of healthcare and a system for effecting the same. The method and system substantially alleviate one or more of the above-described problems with existing healthcare management programs. In one embodiment, the invention is a management program which is based upon an outcomes-focused risk sharing model. The model establishes the criteria that form the basis for a relationship between a healthcare provider and a healthcare vendor. The management program is provided by the healthcare vendor to the healthcare provider for a fixed fee or cost.

The management program includes a plan of care that defines protocols to utilize during the provision of care to individuals, documentation tools that provide evidence of the treatment and progress of each individual, and indemnity instruments which distribute the risks associated with the provision of care between the healthcare provider and the healthcare vendor.

The management program is designed to reduce the risk of having adverse outcomes, and the costs of liability associated with those adverse outcomes, during the provision of healthcare. When the healthcare provider complies with the management program as outlined in the plan of care, the healthcare provider is in fact providing a standard of care which cannot be considered negligent. Although some adverse outcomes may occur, the healthcare provider is able to demonstrate, using the documentation tools, that those adverse outcomes occurred while the healthcare provider was complying with nationally accepted standards of care. Even though the healthcare provider may still be susceptible to litigation, the size of any settlement or judgement rendered against the healthcare provider should be greatly reduced when compared to those settlements and judgments involving acts of negligence during the provision of healthcare. Further, if a settlement or a judgement is rendered against the healthcare provider based on an adverse outcome that occurred during a period of compliance with the management program, the indemnity instruments provide up to a set amount of money to offset liabilities associated with that settlement or judgement.

Other features of the invention will become apparent by consideration of the detailed description and accompanying drawings.

### BRIEF DESCRIPTION OF THE DRAWINGS

In the drawings:

FIG. 1 schematically illustrates a relationship between a healthcare provider and a healthcare vendor for the provision of care as defined by a management program of the invention.

FIG. 2 schematically illustrates components of the management program of FIG. 1.

FIG. 3 illustrates a wound treatment protocols chart of the management program of FIG. 1.

FIG. 4 illustrates a documentation website of the management program of FIG. 1.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

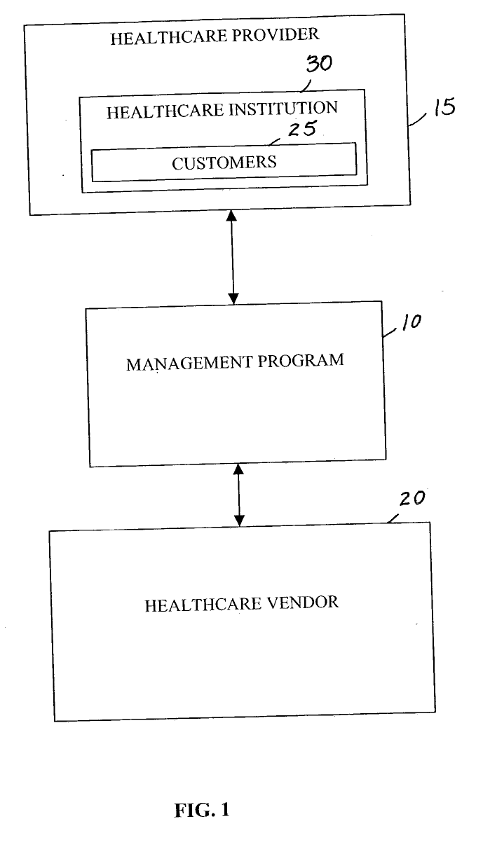

Described below is a skin and wound care management program 10 embodying the invention. While the management program of the invention is described in the context of skin and wound care management, the management program can be easily applied to the provision of any healthcare services, without regard to the length or nature of care provided, or type of illness treated. The skin and wound care management program 10 defines a relationship between a healthcare provider 15 and a healthcare vendor 20 for the provision of care to customers 25 (e.g., residents, patients, and the like) of a healthcare institution 30 managed by the healthcare provider 15 (see FIG. 1).

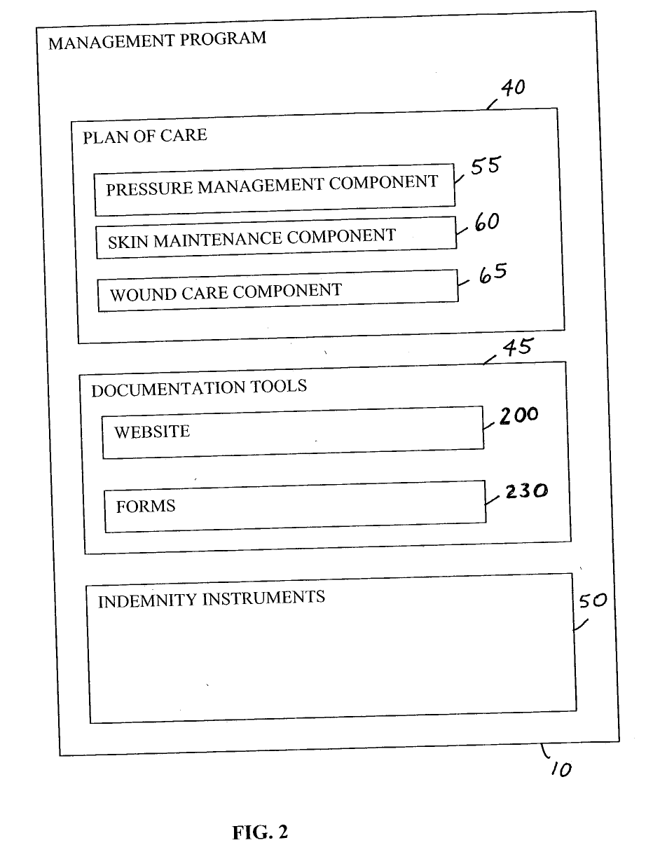

The management program 10 is designed to reduce the risk of having adverse outcomes, and the costs of liability associated with those adverse outcomes, during the provision of healthcare. Employees of the healthcare institution 30 practice the management program 10 using protocols defined in a plan of care 40 (see FIG. 2) provided by the healthcare vendor 20. The plan of care 40 is designed to ensure that every customer 25 of the healthcare institution 30 is provided with a consistent level of care, in a manner that is compliant with accepted national standards of care. The management program 10 further includes documentation tools 45 that provide evidence of the treatment and progress of each customer 25, and the clinical oversight by healthcare professionals of such treatment and progress. When the healthcare provider 15 complies with the management program 10 as outlined in the plan of care 40, the healthcare provider 15 by definition is providing a standard of care which cannot be considered negligent. Although some adverse outcomes may occur, the healthcare provider 15 is able to demonstrate, using the documentation tools 45, that those adverse outcomes occurred while the healthcare provider 15 was complying with accepted national standards of care. Even though the healthcare provider 15 may still be susceptible to litigation, the size of any settlement or judgement rendered against the healthcare provider 15 should be greatly reduced when compared to those settlements and judgments involving acts of negligence during the provision of healthcare. Further, the management program 10 includes indemnity instruments 50 that operate to distribute the risks associated with the provision of care between the healthcare provider 15 and the healthcare vendor 20.

Implementation of the management program 10 begins with a baseline evaluation of the healthcare institution 30. The evaluation generates a profile of the healthcare institution or facility 30. The profile includes facility information (e.g., name, shipping address, mailing address, phone, fax, email), employee information (e.g., name, position, responsibilities, contact information), current census information (e.g., the number of customers 25 admitted to the facility on a specific day), and average monthly admission information (e.g., the average number of customers 25, admitted for any length of time, to the facility during a single month where the average is determined over a number of months).

The profile also includes number of licensed beds information (e.g., the number of state licensed beds in the facility), number of Medicare certified beds information (e.g., the number of beds certified for use with Medicare residents), number of beds by type information (e.g., the number of beds utilized in each care area such as skilled, palliative, Alzheimers, sub-acute, assisted/independently living). The profile also includes mattress information (e.g., the kind of mattress utilized on each bed including the cost and remaining life of the mattress), equipment rental information (e.g., the number of rental beds and/or mattresses and the average cost of those rentals), protocol information (e.g., the assessment scales utilized in assessing the customers 25 and existing protocols or policies for wound prevention and treatment), resident information (e.g., the number of customers 25, the RUG of each customer 25), and equipment information (e.g., types of support surfaces, specialty overlays, and other equipment utilized, and the costs and remaining life each type of equipment). The profile also includes skin care product information (e.g., types of moisturizers, incontinence care products, non-prescription specialty topicals, and other products utilized, and the costs and remaining inventory of same), other cost information (e.g., costs for outside consulting services, educational tolls, continuing education unit (“CEU”) programs, procurement, product evaluations, meetings with representatives of products and services, inventory management, product in-service), and documentation information (e.g., existing documentation tools).

Based on the evaluation, a plan of care 40 is established specifically for the healthcare institution 30. The plan of care 40 is established using a predetermined standard set of best care protocols (e.g., protocols that are standardized based on guidelines provided by the Agency for Healthcare Research and Quality (“AHRQ”)). In some embodiments, the plan of care 40 may incorporate special protocols requested by the healthcare provider 15. However, deviations from the standard set of best care protocols must be verified to ensure continued compliance with national standards of care. The plan of care 40 is generally broken down into three main program components: a pressure management component 55, a skin maintenance component 60, and a wound care component 65.

The pressure management component 55 provides the healthcare institution 30 a fixed selection of clinically effective therapeutic pressure management support surfaces and seating products. Utilization of the pressure management surfaces and seating products is intended to prevent the incidence of pressure wounds (e.g., pressure ulcers) and assist in the treatment of existing pressure wounds and/or wounds without pressure etiology. A number of product selection trees (not shown) may be provided to assist employees of the healthcare institution 30 in determining which therapeutic pressure management support surfaces and/or seating products to utilize for the provision of care to the customers 25 of the healthcare institution 30.

An example of a support surface product selection tree is described where the pressure management support surfaces include TEMPUR-MED brand mattresses, TEMPURAP brand mattresses, TEMPURAIR brand mattresses, and TEMPUR-PLUS3 brand mattress, all of which are provided by Tempur-Medical, Inc. of Lexington, Ky. TEMPURAP, TEMPURAIR, and TEMPUR-PLUS3 are all trademarks of Tempur World, Inc of Lexington, Ky. TEMPUR-MED is a registered trademark of Dan Foam A/S Corporation of Denmark.

When assessing an customer 25 using the example support surface product selection tree, the employee first determines whether the customer 25 has a wound. If the customer 25 does not have a wound, a risk intervention path is followed. If the customer 25 does have a wound, a treatment path is analyzed.

The risk intervention path utilizes a risk factor score (e.g., a score based on the Braden Scale, a score based on the Norton+ Scale) as a determination of a risk factor. The risk factor may be a first risk factor (e.g., a Braden score greater than 16, a Norton+ score greater than 15), a second risk factor (e.g., a Braden score between 10 and 16, a Norton+ score between 10 and 15), or a third risk factor (e.g., a Braden score less than 9, a Norton+ score less than 10). If the customer 25 falls within the first risk factor, the employee places a TEMPUR-MED brand mattress on the customer's bed and establishes a turning schedule for the customer (e.g., turn the customer every two hours). If the customer 25 falls within the second risk factor, the employee places a TEMPUR-MED brand mattress on the customer's bed and places seating products on chairs utilized by the customer 25 so the customer does not go more than a predetermined time frame (e.g., 20 minutes) without pressure management. Similarly, the employee establishes a turning schedule for the customer 25. If the customer 25 falls within the third risk factor, the employee places a TEMPURAP brand mattress on the customer's bed and places seating products on chairs utilized by the customer 25 so the customer does not go more than a predetermined time frame without pressure management. Further, the employee obtains a skin/wound consult to determine if further protocols should be implemented. A skin/wound consult is obtained by contacting a specialist as discussed further below. Similarly, the employee establishes a turning schedule for the customer 25.

The employee monitors the customer 25 and documents any interventions performed while the customer 25 is treated using the above defined risk factor. If the interventions performed generate acceptable results, the process of monitoring and documenting continues. If the interventions performed do not generate acceptable results, the employee determines if the customer 25 has developed a wound. If a wound has not developed, the employee advances the customer to the next severity of risk factor (e.g., move from the first risk factor to the second risk factor, move from the second risk factor to the third risk factor), and proceeds as discussed above. If a wound has developed, the employee obtains a skin/wound consult and utilizes the treatment path discussed below to assess the customer 25.

The treatment path begins with a determination of the type of wound (e.g., a pressure wound, a non-pressure wound). If the customer 25 has a pressure wound, the employee determines the stage (e.g., stage I/II, stage III/IV) of the wound based on the depth of the wound and continues according to the classification. If the wound is a stage I/II or stage III/IV wound, the employee places a TEMPUR-MED brand mattress on the customer's bed and places seating products on chairs utilized by the customer 25 so the customer does not go more than a predetermined time frame without pressure management. Further, the employee follows all appropriate skin and wound protocols as discussed further below. If the wound is a stage III/IV wound, the employee also implements a multi-disciplinary care plan including nutritional, rehabilitation, and pharmacological consultations.

Similar to the risk intervention path, the employee establishes a turning schedule for the customer 25, monitors the customer 25, and documents any interventions performed while the customer 25 is treated. If the interventions performed generate acceptable results, and the employee and/or a clinician believes circumstances warrant, the employee can consider changing the status of the customer 25 to the next lower acuity surface. If the interventions performed do not generate acceptable results, and the employee and/or a clinician believes circumstances warrant, the employee can consider changing the status of the customer 25 to the next higher acuity surface. If the customer 25 is provided with the TEMPURAP brand mattress, the TEMPURAIR brand mattress, or the TEMPUR-PLUS3 brand mattress, the employee ensures the care plan remains appropriate and continues to monitor the customer 25 to determine whether the current support surface is appropriate. If the customer 25 is utilizing the TEMPUR-PLUS3 brand mattress and the interventions performed are not generating acceptable results, a skin/wound consult is obtained.

If the customer 25 has a non-pressure wound, the employee determines the type (e.g., ischemic, surgical, or stasis ulcer; flap/graft) of the wound based on the etiology of the wound and continues according to the classification. If the wound is a ischemic, surgical, or stasis ulcer wound, the employee develops a care plan to address the underlying disease processes and then continues as if the customer 25 has a stage I/II pressure wound. If the wound is a flap/graft wound, the employee determines if the flap is intact. If the flap is intact, the employee continues as if the customer has a stage I/II pressure wound while making sure to take shear precautions when turning the customer. If the flap is not intact, the employee obtains a skin/wound consult.

A seating product selection tree can similarly be described where the seating products include VISCORIDE brand cushions, TEMPUR-MEDICAL brand therapy pads, TEMPUR-MEDICAL brand flat and curved base wheelchair cushions, and TEMPUR-MEDICAL brand cushions for geriatric chairs, recliners, and chairs, all of which are provided by Tempur-Medical, Inc. of Lexington, Ky. VISCORIDE and TEMPUR-MEDICAL are trademarks of Tempur World, Inc. of Lexington, Ky. In one embodiment, the decisions in the selection tree are based on the functional ability of the customer (e.g., ambulatory, chair/bed bound), the customer's ability to weight shift or reposition while seated, the customer's use of a chair walker, and the presence of pressure ulcers. In other embodiments, the decisions in the selection tree are based on other factors found to be important in the choice of seating products.

The example product selection trees described are for purposes of illustration only and are not intended to limit the types of decisions and/or products addressed by other product selection trees.

In one embodiment, the healthcare institution is provided with a therapeutic mattress for each bed in the healthcare institution 30. Mattresses for higher acuity customers (e.g., powered mattresses) are initially provided on a current census basis and inventory control practices are utilized to control the number and types of mattresses available at the healthcare institution 30. If an emergency arises, the healthcare provider 15 contacts the healthcare vendor 20 to determine how to proceed.

The skin maintenance component 60 provides the healthcare institution 30 with a comprehensive inventory of high quality, non-prescriptive skin care products in quantities necessary to maintain and enhance the skin condition of each customer 25. The inventory generally includes the following skin care products: moisturizers, barrier creams, skin cleansers, anesthetic creams, antibiotic ointments, antihistamine creams, sun blocks, steroidal creams, soaks, antifungal products, and dressings for covering skin tears. A skin care basic formulary chart (not shown) may be provided that lists the types of skin care products (e.g., moisturizers—melting moisturizer cream and fragrance free lotion, and barrier creams—fragrance free barrier ointment with zinc) available to the healthcare institution 30, and the packaging (e.g., 4 ounce tube, and 64 ounce pump bottle) of each type of product. Such a skin care basic formulary chart provides a reference for employees in selecting a type of product to perform a specific protocol. A skin care product usage guide is also generally provided. An example of a skin care product usage guide is illustrated in the following table:

TABLE 1
SKIN CARE PRODUCT USAGE GUIDE

| Item | Function | Indications | Procedure |
|---|---|---|---|
| Moisturizing | Holds water in skin | To repair and | Apply at any time to |
| Cream | for hours to stop | prevent dry or | area at risk, but |
| | dryness. | cracked skin and | ideally immediately |
| | | skin tears on | after washing or |
| | | problem areas. | hydrating, to trap in |
| | | | maximum moisture. |
| Moisturizing | Holds water in skin | Use daily to stop dry | Apply any time, but |
| Lotion | hours longer than | skin and prevent | ideally immediately |
| | other moisturizers to | complications on all | after washing or |
| | stop dry skin, | parts of the body. | hydrating, to trap in |
| | | | maximum moisture. |
| Barrier Ointment | Moisture barrier to | Used as needed to | 1. Remove any urine |
| | prevent irritation | prevent and help | or fecal matter. |
| | from incontinent | heal irritation from | 2. Apply a generous |
| | episodes. | fecal matter, urine, | amount to affected |
| | | moisture and | area. |
| | | abrasion. | 3. Repeat after every |
| | | | incontinent episode or |
| | | | as needed. |
| Cleansing Foam | To gently remove | To gently clean and | 1. Shake can well. |
| urine and fecal | moisturize skin after | 2. Direct spray | |
| matter and clean | incontinent | toward area to be | |
| skin, | episodes. | cleansed. | |
| | | | 3. Wipe away foam. |
| | | | 4. Repeat if |
| | | | necessary. |
| | | | 5. No rinsing |
| | | | necessary. |
| Perineal/Ostomy | Gently remove urine | To gently clean and | 1. Direct spray |
| Wash | and fecal matter and | moisturize skin after | toward area to be |
| | clean skin, | incontinent | cleansed. |
| | | episodes. | 2. Wipe away. |
| | | | 3. Repeat if |
| | | | necessary. |
| | | | 4. Does not need to |
| | | | be rinsed. |
| 10% Coal Tar | Used locally, Tar | Used to control | Apply to affected |
| Solution In | derivatives and | psoriasis or eczema | areas one to four |
| Concentrated | concentrated cream | on skin or scalp and | times daily or as |
| Cream Base | base help to correct | to relieve itching | directed by physician. |
| | abnormalities of | and flaking. Melts | |
| kerationization | into skin on contact | | |
| | (loosens cornified | for deeper | |
| epithelium). | penetration and 12 | | |
| | | hour moisturizing | |
| | | power. | |
| 10% Coal Tar | Used as a lotion, Tar | Used to control | Apply to affected |
| Solution In | derivatives help to | psoriasis or eczema areas one to four | |
| Lotion Base | correct abnormalities | on skin or scalp and | times daily or as |
| | of kerationization | to relieve itching | directed by a |
| | (loosens comified | and flaking. Melts | physician. For scalp, |
| | epithelium). | into skin on contact | massage into scalp |
| | | for deeper | before bed, cover |
| | | penetration and 8 | with cap, wash out in |
| | | hour moisturizing | the morning. |
| power. | | | |
| Hydrocortisone | Localized anti- | Temporary relief of | Adults and Children 2 |
| Creams: 1% and | inflammatory | itching associated | years of age and |
| 0.5% | activity that is non- | with minor skin | older: Apply to |
| specific. | irritations, | affected area not | |
| | | inflammation, and | more than 3-4 times |
| | | rashes. | daily. |
| Bacitracin | Topical anti- | Topical antibiotic | Clean affected area. |
| Ointment | infectives may be | preparations are | Apply a small amount |
| | either bactericidal or | used for infection | of this produce (an |
| | bacteriostatic. Most | treatment or | amount equal to the |
| | inhibit protein | prophylaxis in minor | surface area of the tip |
| | syntheses. | cuts, wounds, bums, | of a finger) on the |
| | | and skin abrasions. | area 1 to 3 times |
| | | Also as an aid to | daily. May be |
| | | healing and for the | covered with a sterile |
| | | treatment of | bandage. |
| | | superficial infection. | |
| Triple Antibiotic | Topical anti- | Topical antibiotic | Clean affected area. |
| Ointment | infectives may be | preparations are | Apply a small amount |
| | either bactericidal or | used for infection | of this produce (an |
| | bacteriostatic. Most | treatment or | amount equal to the |
| | inhibit protein | prophylaxis in minor | surface area of the tip |
| | synthesis. | cuts, wounds, burns, | of a finger) on the |
| | | and skin abrasions. | area 1 to 3 times |
| | | Also as an aid to | daily. May be |
| | | healing and for the | covered with a sterile |
| | | treatment of | bandage. |
| | | superficial infection. | |
| Skin | Delivers enzyme | Rich formulation | Apply twice daily or |
| conditioning and | action in a strong | rehydrates and | as needed. For best |
| rehydrating | moisturizing base | moisturizes rough, | results, moisten skin |
| crème/vegetable | which penetrates and | dry skin to soothe | first or apply |
| enzyme base | works for hours. | associated itchiness, | immediately after |
| (Nuvase). | improve appearance | washing or showering | |
| | | and improve texture. | while skin is moist. |
| | | Works quickly on | |
| | | many skin | |
| | | conditions, | |
| | | including those | |
| | | associated with | |
| | | contact dermatitis, | |
| | | cellulitis, sunburn, | |
| | | minor lacerations, | |
| | | venous stasis | |
| | | dermatitis, and areas | |
| | | of skin at risk. | |
| Penetrating and | Penetrates quickly | Use on rough, dry, | Apply twice daily or |
| toning emollient | for rapid relief, | or reddened skin to | as needed. For best |
| with vegetable | Light formation | soothe associated | results, apply as soon |
| enzyme base | seems dry to touch | itchiness, improve | as possible after skin |
| (Provase). | delivers enzymes to | appearance, and | problem occurs. |
| | skin immediately. | improve texture. | |
| | | Works quickly on | |
| | | many skin | |
| | | conditions, | |
| | | including those | |
| | | associated with | |
| | | abrasions, contact | |
| | | dermatitis, cellulitis, | |
| | | and insect bites | |
| | | (mosquitoes, fire | |
| | | ants). | |
| Antihistamine | Provides some local | Temporary relief of | Adults and Children 2 |
| Cream | analgesic activity | itching due to minor | years of age and |
| | and is used to relieve | skin disorders, | older: Apply to |
| | itching. | poison ivy, poison | affected area not |
| | | sumac, and poison | more than 3-4 times |
| | | oak exposure, | daily. Children under |
| | | sunburn, insect bites | 2 years of age: |
| | | and stings. | Consult a physician. |
| Anesthetic | Inhibit conduction of | Topical anesthetics Always when | |
| Cream | nerve impulses from | in local skin | practical, cleanse the |
| | sensory nerves. This | disorders, including | affected area with |
| | action results from | itching and pain due | mild soap and warm |
| | an alteration of the | to minor burns, skin | water, and rinse |
| | cell membrane | manifestations of | thoroughly. Apply to |
| | permeability, | systemic diseases | the affected area 3-4 |
| | | (e.g. chicken pox), | times daily. |
| | | prickly heat, | |
| | | abrasions, sunburn, | |
| | | plant poisonings | |
| | | (e.g. poison ivy), | |
| | | and insect bite. | |
| Miconazole | Alters cellular | Tinea Pedis | Clean the affected |
| Nitrate 2% Anti- | membrane | (athlete's foot) | area and dry |
| fungal Cream | permeability. | Tinea Cruris (jock | thoroughly. Apply a |
| | | itch) and Tinca thin layer of the | |
| | | Corporis | product over affected |
| | | (ringworm), | area twice daily |
| | | | (morning and night) |
| | | | or as directed by a |
| | | | doctor. Supervise |
| | | | children in the use of |
| | | | this product. For |
| | | | athlete's foot: Pay |
| | | | special attention to |
| | | | spaces between the |
| | | | toes; wear well- |
| | | | fitting, ventilated |
| | | | shoes, and change |
| | | | shoes and socks at |
| | | | least once daily. For |
| | | | athlete's foot and |
| | | | ringworm, use daily |
| | | | for 4 weeks; for jock |
| | | | itch, use daily for 2 |
| | | | weeks. If condition |
| | | | persists longer, |
| | | | consult a doctor. |
| | | | This product is not |
| | | | effective on the scalp |
| | | | or nails. |
| Miconazole | Alters cellular | Tinea Pedis | Clean the affected |
| Nitrate 2% Anti- | membrane | (athlete's foot) | area and dry |
| fungal Powder | permeability. | Tinea Cruris (jock | thoroughly. Apply a |
| | | itch) and Tinea | thin layer of the |
| | | Corporis | product over affected |
| | | (ringworm), | area twice daily |
| | | | (morning and night) |
| | | | or as directed by a |
| | | | doctor. Supervise |
| | | | children in the use of |
| | | | this product. For |
| | | | athlete's foot: Pay |
| | | | special attention to |
| | | | spaces between the |
| | | | toes; wear well- |
| | | | fitting, ventilated |
| | | | shoes, and change |
| | | | shoes and socks at |
| | | | least once daily. For |
| | | | athlete's foot and |
| | | | ringworm, use daily |
| | | | for 4 weeks; for jock |
| | | | itch, use daily for 2 |
| | | | weeks. If condition |
| | | | persists longer, |
| | | | consult a doctor. |
| | | | This product is not |
| | | | effective on the scalp |
| | | | or nails. |
| Sun Block 32 | Broad spectrum sun | When applied as | Apply each morning |
| SPF | block and | directed provides | to face and backs of |
| | moisturizer for daily | daily protection | hands as a |
| | use. | from the sun, | combination block |
| | | blocking a very and moisturizer. | |
| | | broad spectrum of | |
| | | harmful UV-A and | |
| | | UV-B rays. | |
| Zinc Oxide, | Provides | Used in the therapy | Refer to product |
| Glycerine, | compressive | of circulatory | insert. |
| Gelatin Bandage, | protection for certain | disturbances of a | |
| 4″ | conditions (see | peripheral nature. In | |
| | indications), may | addition, certain | |
| | also be used to fix a | static conditions of | |
| | primary dressing in | the leg and foot | |
| | place (refer to | which may be | |
| | product insert), | treated with | |
| | | | compressive- |
| | | | correcting dressings |
| | | | such as post-fracture |
| | | | foot conditions, |
| | | | spastic weak foot |
| | | | conditions |
| Astringent | Provides soothing | For temporary relief | One tablet dissolved |
| Solution Tablets | relief of minor skin | of minor skin | in 12 ounces of water |
| | irritations, | irritations due to | makes a modified |
| | | poison ivy, poison | Burrow's Solution |
| | | oak, poison sumac, | approximately |
| | | insect bites, athlete's | equivalent to a 1:40 |
| | | foot or rashes | dilution; two tablets, |
| | | caused by soaps, | a 1:20 dilution, and |
| | | detergents, | four tablets, a 1:10 |
| | | cosmetics or | dilution. Dissolve |
| | | jewelry. | one or two tablets in |
| | | | water and stir the |
| | | | solution until fully |
| | | | dissolved. Do not |
| | | | strain or filter the |
| | | | solution. Can be used |
| | | | as a compress, wet |
| | | | dressing or as a soak. |
| | | | AS A COMPRESS |
| | | | OR WET |
| | | | DRESSING: |
| | | | Saturate a clean, soft, |
| | | | white cloth (such as a |
| | | | diaper or torn sheet) |
| | | | in the solution; gently |
| | | | squeeze and apply |
| | | | loosely to the affected |
| | | | area. Saturate the |
| | | | cloth in the solution |
| | | | every 15 to 30 |
| | | | minutes and apply to |
| | | | affected area. |
| | | | Discard solution after |
| | | | each use. Repeat as |
| | | | often as necessary. |
| | | | AS A SOAK: Soak |
| | | | affected area in the |
| | | | solution for 15 to 30 |
| | | | minutes. Discard |
| | | | solution after each |
| | | | use. Repeat 3 times a |
| | | | day. |
| Topical | Protects and | For use as a wound | Clean wound |
| Ointment with | insulates wound site, | dressing to manage | thoroughly and dry |
| Plant Extracts | provides | pressure ulcers, | surrounding skin, |
| | environment for | stasis ulcers, apply a thin layer of | |
| | healing to take | diabetic skin ulcers, | ointment directly on |
| | place. | skin irritations, cuts, | wound surface |
| | | abrasions and skin | extending over onto |
| | | irritations associated | the intact skin a |
| | | with periostomal | minimum of 1-2 |
| | | care. | times daily. Apply a |
| | | | bandage to cover the |
| | | | wound after |
| | | | application of the |
| | | | ointment. If gauze is |
| | | | used as a cover |
| | | | dressing, it should be |
| | | | moistened before |
| | | | application. (Refer to |
| | | | product insert.) |
| Wound Closure | Adhesive closure | Use as a primary | (Refer to protocols |
| Strips | device for non- | wound closure (refer | and product insert.) |
| | invasive closure of | to protocols). | |
| | superficial wounds. | | |
| Film Dressings | Protects site while | For use as a primary | (Refer to protocols |
| | allowing for moist | or secondary | and product insert.) |
| | wound environment, | dressing for non- | |
| | | draining wounds | |
| | | (refer to protocols). | |
| Thin Foam | Protects and | For use as a primary | (Refer to protocols |
| | insulates site. | or secondary | and product insert.) |
| | | dressing over an | |
| | | absorptive wound | |
| | | filler (refer to | |
| | | protocols). | |
| Topical Wound | Topical | For use as a topical | (Refer to protocols |
| Ointment | Agent/Wound filler | agent/would filler in | and product insert.) |
| | | stage II wounds. | |

The skin care product usage guide shown in Table 1 provides employees of the healthcare institution 30 information regarding the function, indications, and procedures for application of each product. In some embodiments, the skin care product usage guide includes an illustration of the product for easy reference.

The wound care component 65 provides the healthcare institution 30 a comprehensive inventory of high quality, non-prescriptive wound management materials in quantities necessary for treating any stage or severity of wound. The inventory generally includes alginates, antimicrobial activity dressings, closure strips, compression/support/tube bandages, contact layers, films, foam dressings, gauze/sponges, hydrocolloids, hydrofibers, amorphous and sheet hydrogels, other absorptives, and wound fillers. A wound care classification and cross reference chart (not shown) may be provided that lists the brand names of wound management materials available to the healthcare institution 30, and the manufacturer of each material. Such a wound care classification and cross reference chart provides a reference for employees in selecting a brand of wound management material to perform a specific protocol.

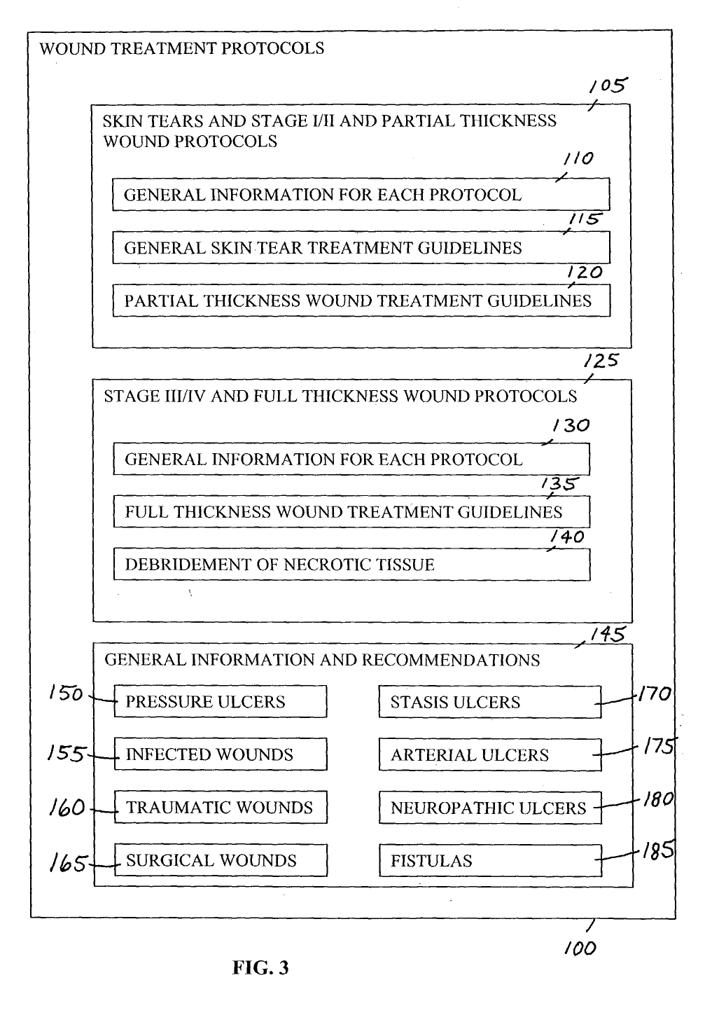

A wound treatment protocol chart is also generally provided. An example of a wound treatment protocol chart 100 is schematically illustrated in FIG. 3. The wound treatment chart 100 provides a hierarchical reference for employees of the healthcare institution 30 when determining the proper wound treatment protocol to follow during the provision of care to an customer 25. The employee first determines what type of wound the customer 25 has (e.g., skin tear, stage I/II wound, stage III/IV wound, partial thickness wound, full thickness wound). The employee is reminded on the chart 100 that the following protocols are for topical treatment only, that a skin/wound consult is required prior to placing any resident on a powered surface, that all reportable pressures (e.g., stage II, III, or IV) must be documented using the documentation tools 45, and that in the event that wounds fail to improve or if the employee has questions, the healthcare vendor 20 should be contacted.

If the customer has a skin tear, a stage I/II wound, or a partial thickness wound, the skin tears and stage I/II and partial thickness wound protocols section 105 of the chart 100 is utilized. The skin tears and stage I/II and partial thickness wound protocols section 105 includes a general information for each protocol portion 110, a general skin tear treatment guidelines portion 115, and a partial thickness wound treatment guidelines portion 120.

The general information for each protocol portion 110 includes treatment guidelines to follow for each wound type in the section 105. The treatment guidelines include the following: (1) cleanse wound with saline or approved wound cleanser, (2) gently blot surrounding dry tissue, (3) use barrier cream on surrounding skin to help prevent maceration if necessary, and (4) elevate area if possible and adjust turning schedule to minimize pressure.

The general skin tear treatment guidelines portion 115 includes treatment guidelines to follow for skin tear wounds. The treatment guidelines include the following: (1) avoid use of tapes or products with adhesives, (2) if wound edges are approximate do not remove until loosened or healed, and (3) secure approximated wound edges with closure strips. If these treatment guidelines do not generate effective results, the general skin tear treatment guidelines portion 115 suggests utilizing the stage II or partial thickness wounds with no to light drainage protocol discussed below.

The partial thickness wound treatment guidelines portion 120 includes protocols for stage I, stage II or partial thickness wounds with no to light drainage, and partial thickness wounds with moderate to heavy drainage. The employee selects one of three classifications of wounds and follows the treatment guidelines for the selected protocol.

For a stage I wound, the treatment guidelines include the following: (1) elevate the area if possible and adjust turning schedule to minimize pressure, (2) moisturize areas each shift by lightly rubbing (no deep massage), apply thin foam and change every five days or when thin foam loosens.

For a stage II wound or a partial thickness wound with no to light drainage, the treatment guidelines include the following: (1) elevate the area if possible and adjust turning schedule to minimize pressure, (2) apply topical products in areas of incontinence (use products usage guide), secondary covering usually not necessary, (3) apply thin foam dressing in areas of incontinence and secure with tape, roll gauze and/or tubular elastic gauze if necessary, change every 3-5 days, and (3) apply thin hyrocolloid in areas of incontinence and change every 3-5 days.

For a stage II wound or a partial thickness wound with moderate to heavy drainage, the treatment guidelines include the following: (1) elevate the area if possible and adjust the turning schedule to minimize pressure, (2) apply topical agent daily in areas prone to contact with moisture (use product usage guide), secondary covering usually not necessary, (3) cover area with gauze, or multi-layered absorptive pad, secure with tape, roll gauze, or tube gauze, change daily, and (4) apply thin foam dressing to area, change daily.

If the customer has a stage III/IV wound or a full thickness wound, the stage III/IV and full thickness wound protocols section 125 of the chart 100 is utilized. The full thickness wound protocols section 125 includes a general information for each protocol portion 130, a full thickness wound treatment guidelines portion 135, and a debridement of necrotic tissue portion 140.

The general information for each protocol portion 130 includes treatment guidelines to follow for each wound type in the stage III/IV and full thickness wound protocols section 125. The treatment guidelines include the following: (1) cleanse wound with saline or approved wound cleanser, (2) gently blot surrounding dry tissue, and (3) use barrier cream on surrounding skin to help prevent maceration if necessary.

The full thickness wound treatment guidelines portion 135 includes protocols for stage III/IV or full thickness wounds with no to light draingage, and stage III/IV or full thickness wounds with moderate to heavy drainage. The employee selects one of two classifications of wounds and follows the treatment guidelines for the selected protocol.

For a stage III/IV wound or a full thickness wound with no to light drainage, the treatment guidelines include the following: (1) elevate the area if possible and adjust turning schedule to minimize pressure, (2) apply amorphous hydrogel, or other appropriate wound filler, if applicable, cover with gauze or absorptive secondary dressing and secure with tape, roll gauze, or tube gauze daily, (3) apply impregnated gauze, cover with gauze or absorptive secondary dressing, or film, secure with tape, roll gauze, or tube gauze, change every 3 days, if additionaly packing is needed, lightly pack with saline moistened gauze, and (3) apply sheet hydrogel or hydrocolloid dressing (use hydrocolloid only on clean, healing stage III ulcers), depending on the amount of exudate, change every 3 days.

For a stage III/IV wound or a full thickness wound with moderate to heavy drainage, the treatment guidelines include the following: (1) elevate the area if possible and adjust turning schedule to minimize pressure, (2) lightly pack with alginate, if additional packing is needed, lightly pack with normal saline moistened gauze, cover with gauze or foam and secure with tape, roll gauze, or tubular elastic gauze, change daily, (3) apply granular wound filler (if tunneling and base is not visible, do not use granular wound filler), if additional packing is needed, lightly pack with saline moistened gauze, cover with gauze or multi-layered non-adherent absorptive pad or foam, secure with tape, roll gauze, or tubular elastic gauze daily, and (4) if ulcer contains slough or is infected, lightly pack with hypertonic saline impregnated dry gauze, cover with gauze or multi-layered non-adherent absorptive pad, secure with tape, film, roll gauze, or tubular elastic gauze, change based on amount of drainage.

The debridement of necrotic tissue portion 140 includes treatment guidelines to follow for debridement of necrotic tissue. The treatment guidelines include the following: (1) if the wound is covered with hard, dry eschar, has no odor or drainage, and is firmly attached, a physician should be consulted to determine if assisted debridement is appropriate, and (2) if no debridement is ordered, cover area with gauze and secure with tape, roll gauze, or tube bandage and change daily, monitor each day and report any changes to physician. Following is table that discusses procedures and notes for the types of debridement:

TABLE 2
DEBRIDEMENT GUIDE

| Type | Procedure | Notes |
|---|---|---|
| Autolytic | Use of moist wound | Cover area with impervious dressing |
| | healing concepts to | or use hydrogel to donate moisture to |
| | soften and “self- | necrotic tissue. |
| | digest” necrotic/ | Monitor each day and report changes |
| | devitalized tissue. | to physician. |
| | | Can be performed independently of |
| | | stage III, stage IV, or full thickness |
| | | wound protocols. |
| Mechani- | Removal of necrotic | Utilize wet-to-dry dressings. |
| cal | tissue using physical | Utilize hydrotherapy (e.g., whirlpool, |
| | force | pulsed lavage) |
| Chemical | Use of enzymatic | Enzymatic debriding agents are |
| | debriding agent to | generally not over-the-counter and |
| | break down | are therefore not included in the |
| | necrotic tissue. | |
| | | fixed cost of the management program. |
| Sharp/ | Use of scalpel, | Must be performed by a licensed |
| | scissors, or | |
| Surgical | other sharp | practitioner certified to perform |
| | instrument to | |
| | remove devi- | debridement. |
| | talized tissue. | If not peformed by a physician, |
| | | consult appropriate State Practice Act. |

If the employee is unsure of what type of wound the customer 25 has or needs additional information about a wound, the employee can utilize the general information and recommendations section 145 of the chart 100. The general information and recommendations section 145 includes information and recommendations about pressure ulcers 150, infected wounds 155, traumatic wounds 160, surgical wounds 165, stasis ulcers 170, arterial ulcers 175, meuropathic ulcers 180, and fistulas 185, as shown in the following table:

TABLE 3
WOUND GENERAL INFORMATION AND RECOMMENDATIONS

| Wound Type | General Information and Recommendations |
|---|---|
| Pressure | Use protocols according to stage and characteristics of |
| Ulcers | wound as noted on assessment. |
| | Identify and address risk/contributing factors such as |
| | pressure, friction, shear, nutrition, moisture, immobility, |
| | sensory, perception, medication regime, and psychosocial |
| | status. |
| Infected | Consult physician for appropriate treatment orders. |
| Wounds | Consider daily wound care using dry hypertonic saline |
| | impregnated gauze dressings. |
| | Avoid the use of occlusive dressings, such as films, foams |
| | with occlusive backing, hydrocolloids, and sheet hydrogels. |
| Traumatic | Select treatment protocols consistent with wound depth and |
| Wounds | characteristics as noted on assessment. |
| Surgical | Approximated (wound edges together) - for topical |
| Wounds | treatment, see stage II and partial thickness wound |
| | protocol. |
| | Open (dehisced, not approximated) - for topical treatment, |
| | see stage III/IV and full thickness wound protocols. |
| | Partial thickness (e.g., skin grafts, donor sites) - request |
| | advanced wound specialist consult. |
| Stasis | For topical treatment, see stage III/IV and full thickness |
| Ulcers | wound protocols. |
| | Identify, assess, and treat underlying conditions and risk |
| | factors (e.g., deep vein thrombosis, smoking, CHF, |
| | mobility, nutrition). |
| | Consider compression, if appropriate (evaluate for cellulitis |
| | and arterial disease). |
| | Consider elevation of extremity (again, evaluate arterial |
| | status). |
| Arterial | For topical treatment, see stage III/IV and full thickness |
| Ulcers | wound protocols. |
| | Identify, assess, and treat underlying conditions and risk |
| | factors (e.g., smoking, diabetes, hypertension, |
| | atherosclerosis, history of vascular procedures/surgeries). |
| | Do not perform compression without thorough examination |
| | of arterial status. |
| | If hard, dry, firmly attached eschar is present with no signs |
| | of infection noted, ask physician if debridement is |
| | appropriate (see debridement of necrotic tissue). |
| | Minimize elevation of lower extremities, consider a |
| | dangling schedule. |
| Neuropathic | For topical treatment, see stage III/IV and full thickness |
| Ulcers | wound protocols. |
| | Identify, assess, and treat underlying conditions and risk |
| | factors (e.g., diabetes, spinal cord injuries, smoking). |
| | Monitor for callous formation around wound edges. |
| | Closely monitor for sources of continued trauma (e.g., fit of |
| | shoes/booties). |
| Fistulas | Consider use of stage III/IV and full thickness wound |
| | protocols. |
| | If drainage is excessive or requires more than daily change |
| | using protocol, consider use of a drainage collector. This |
| | could be a wound manager or an ostomy appliance. Obtain |
| | an advanced wound care nurse consultation. |

The plan of care 40 described above is for purposes of illustration only and is not intended to limit the types of protocols addressed by other plans of care or components thereof.

The documentation tools 45 of the management program 10 are designed to provide the healthcare provider 15 the information needed to provide continuing care to a customer 25, to allow the healthcare vendor 20 to monitor the level of compliance with the management program 10 by the healthcare provider 15, and to provide verified data that employees of the healthcare institution 30 have provided the customers 23 with a nationally accepted standard of care, while being monitored by healthcare professionals associated with the healthcare vendor 20.

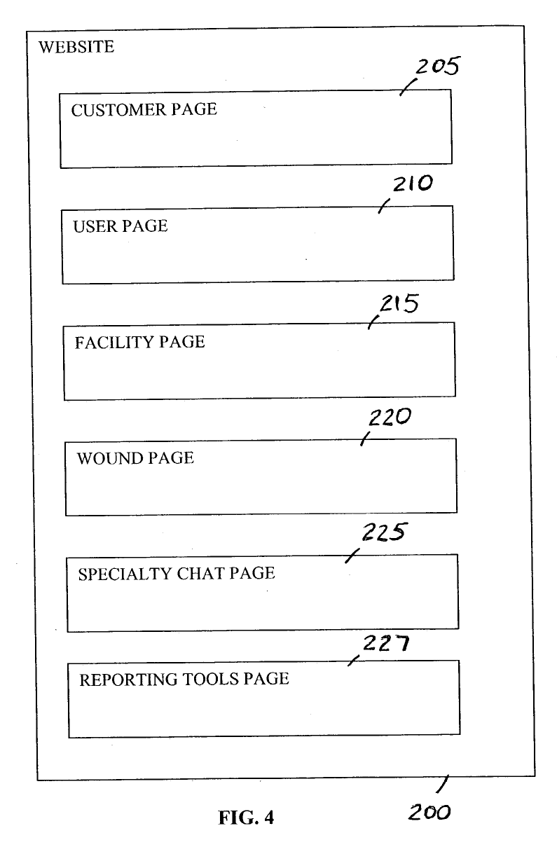

The documentation tools 45 includes a Health Insurance Portability and Accountability Act (“HIPAA”) compliant website 200 (FIG. 2) that allows for the monitoring of the management program 10 and the tracking of outcomes. A user accesses the website by using a login name and a password. A user is only allowed to access the pages of the website if the user is defined by a user profile that affords them privileges to use the website. A user profile includes information regarding the usage rights of the user, the name of the user, the healthcare institution 30 at which the user works, a fax number, a telephone number, a user name, and a password. These user profiles can be updated by users with appropriate authorization (e.g., an administrator at the healthcare institution 30 at which the user works). Once access is gained to the site, the user can select from a number of pages depending upon the user's level of security access. As shown in FIG. 4, in one construction, the pages include an customer page 205, a user page 210, a facility page 215, a wound page 220, a specialist chat page 225, and a reporting tools page 227.

On the customer page 205, the user can view information regarding a profile of a selected customer 25, update information regarding the selected customer 25, and/or view history information about the selected customer 25. Information in the selected customer's profile may include the selected customer's name, gender, date of birth, date of admission to the healthcare institution 30 at which they are currently located, social security number, weight, height, identification number, room number, place of service, attending physician, previous location, primary contact, current equipment and/or consumables, past equipment and/or consumables, diagnosis information, observations and other factors, and the like. History information may include information regarding assessments performed on the selected customer 25 including the assessment date, location, the type of wound or condition of the selected customer 25, the origin and size of the wound or condition, a detailed description of the wound, and comments on wound care activities and observations. The user can edit the information if necessary.

The wound page 225 allows the user to select a specific wound for a specific customer 25. Once a wound is selected, information regarding the wound similar to the historical information is displayed. A pictorial log of the wound may be provided which allows for an accurate record of what the wound looked like throughout the treatment process.

The facility page 215 includes information regarding healthcare institutions 30 that utilize the management program 10. Each facility page 215 includes information such as the contact at the facility, the facility's name, the number of beds at the facility, the types of beds at the facility (e.g., skilled beds, swing beds, Alzheimer's, sub-acute, rehab, palliative), the address, phone number and fax number of the facility, the owner of the facility, the affiliation of the facility (e.g., ownership of the facility, management of the facility), the special areas of care and services provided by the facility (e.g., Alzheimer's, wound care, rehab, ventilator, physical therapy, occupational therapy, and hospice), the resources of the facility (e.g., in-house or captive pharmacy, wound care consultants, nutritional consultants, wound care team, and internet access), the average number of daily rental beds and/or mattresses used, the risk scale used (e.g., Braden Scale, Norton+ Scale), and other essential information relating to the facility.

The specialist chat page 225 allows the user to communicate with a healthcare professional or specialist that may be able to provide information above and beyond that available on the protocols. Such information may assist the healthcare provider in obtaining a more beneficial result with respect to a special situation. Generally, information necessary to perform a consult must be entered by the user before the communication with the healthcare professional. A time for the consult may be setup by contacting the healthcare vendor 20. At the scheduled time the user can communicate with the healthcare professional using the specialist chat page 225. All documentation regarding recommendations made by the wound specialist must be placed in the website 200 during the interactive specialist chat. In one embodiment, skin/wound consults are obtained using the specialist chat page 225. In other embodiments, skin/wound consults and other types of consults are alternatively obtained (e.g., in person, via telephone, via fax, and via e-mail).

The reporting tools page 227 allows the user to generate reports for use in the management of the customers 25 and/or the management of quality at the healthcare institution 30. The reports may be reviewed on the website 200 and/or output for later use. The reporting tools page 227 allows the user to easily access and analyze data that has been collect on the website 200. The user may generate predefined reports or self-defined reports. The reports may include data from a single healthcare institution 30 or from multiple healthcare institutions 30. The types of reports the user may generate depends on the user's access level. The reports generally may be defined for a specified period of days, weeks, or months. Further, the reports may also be defined by the user to display only those parameters the user desires to review. In one embodiment, the user may generate a summary report. The parameters of the summary report may include the number of customers 25 having wounds, the average risk assessment score of those wounds, the number of wounds acquired in the healthcare institution 30, the number of wounds inherited on admission of the customer 25 to the healthcare institution 30, and the number of wounds of unknown origin. The parameters may further include specific information about currently active wounds such as the number of active pressure wounds, the number of wounds on first time customers 25, the number of new wounds on existing customers 25, and the number of wounds indicated as healed. The specific information about currently active wounds may be broken down based on the severity of the wound (e.g., stage I, stage II, stage III, stage IV, no stage) and the type of wound (e.g., pressure wound, stasis wound, surgical wound, other wound). Other parameters may include the aggregate wound size of all wounds on customers 25 at the healthcare institution 30, the percent change in aggregate wound size at the healthcare institution 30, the date of the last customer update, and the date of the last photo upload. In other embodiments, the user can generate reports for use in analyzing dynamics of the customers 25. For example, the user may generate a skin care action team report that lists the name of each customer 25 that has a wound and the type, severity, and location of that wound. Alternatively, the user can generate other reports that focus on customers with wounds (e.g., a customers with wounds report, a customers with wounds by admission score report, a customers with wounds by origin report, a customers with wounds by type of ulcer report, a customers with wounds by location report, a customers with wounds without improvement report, a customers with high risk wounds report, and a residents with healed wounds report). In other embodiments, the user may generate an incidence report that provides information regarding incidence that occurred at the healthcare institution 30 for a specified period. Each incidence may be classified based on the severity of the incidence (e.g., any incidence, significant incidence). Any incidence may be defined as an occurrence of a pressure ulcer of any stage. Significant incidence may be defined as an occurrence of a full thickness wound or the second occurrence within a one-month period of a stage II ulcer. The incidence may be reported as a number and/or a percentage with respect to the census of the healthcare institution 30, or any subgroup of the census (e.g., customers 25 admitted during a certain period). In other embodiments, the user may generate other customer reports for use in monitoring the management program 10 and/or tracking of outcomes.

The website 200 provides the user alerts prompting the user to enter information necessary for the use of the management program 10 at the appropriate time. Generally, wound information must be entered at least every week. The website 200 may automatically monitor the information entered or the healthcare vendor 20 may manually observe the information available to determine whether the healthcare provider 15 is complying with all applicable protocols. The healthcare provider 15 is encouraged to comply with the requirements of the website 200 because if information is not entered appropriately, the indemnity instruments 50 may not apply for reasons of non-compliance.

If the healthcare vendor 20 determines the healthcare provider 15 is not compliant with requirements of the management program 10, a notification of non-compliance may be issued. The notification informs the healthcare provider 15 of the deficiency, provides an amount of time to correct the deficiency, and communicates the loss of indemnity coverage for the period of non-compliance if the deficiency is not cured as outlined in the notification.

The documentation tools 45 may also include paper-based forms that allow employees, clinicians, and other healthcare professionals to record information similar to that entered in the website 200. The paper-based forms 230 allow employees of the healthcare institution 30 to accurately record information for later entry on the website 200. The paper-based forms 230 also provide a ready reference for use in continuing care to an customer 25.

The documentation tools 45 described above are for purposes of illustration only and are not intended to limit the types of documentation utilized in the management program 10. In other embodiments, other types of documentation tools 45 (e.g., e-mail, fax, handheld computing devices) are utilized.

The indemnity instruments 50 of the management program 10 distribute the risks associated with the provision of care between the healthcare provider 15 and the healthcare vendor 20. The indemnity instruments 50 provides up to a set amount of money (e.g., $250,000) to be paid per incident if there is for a settlement or judgement resulting from a failure of the management program 10, and if the failure occurred while the healthcare provider 15 was complying with the plan of care 40. The documentation tools 45 of the management program 10 are available if needed to demonstrate such compliance.

In one embodiment, an agreement is formed between the healthcare vendor 20 and the healthcare provider 15. If the management program 10 fails, the healthcare vendor 20 provides the healthcare provider 15 up to the set amount of money as a result of or in response to the adverse outcome. This money can then be utilized by the healthcare provider 15 to pay a deductible, or a portion thereof, for an insurance policy which also covers the adverse outcome. If the settlement or judgement is not large enough to warrant a claim to the insurance policy, the monies received by the healthcare provider 15 from the healthcare vendor 20 can be utilized to directly pay for the settlement or judgement. This relationship is a type of co-insurance.

In another embodiment, an agreement is formed between the healthcare vendor 20, the healthcare provider 15, and an insurance company. If the management program 10 fails, the healthcare vendor 20 provides the insurance company up to the set amount of money as a result of or in response to the adverse outcome. Although this money cannot be utilized by the healthcare provider 15 to offset the deductible it must pay the insurance company, the agreement often results in decreased premiums and/or deductibles for the healthcare provider 15. This relationship is a type of re-insurance. Essentially, the insurance company is benefited through a reduction in the amount of risk it must assume with respect to the insurance policy provided to the healthcare provider 15. Of course, other types of indemnity instruments 45 are considered to be within the scope of the invention.

The healthcare vendor 20 provides the healthcare provider 15 the management program 10 for a fixed fee. The contract establishing the financial obligations of the relationship is developed in accordance with actuarial models that are designed to analyze the costs associated with providing a comprehensive solution. By looking at historical data for similar situations, the healthcare vendor is able to establish pricing similar to that devised for PPS type situations. The overall cost of the management program 10 is often close to the price the healthcare provider 15 is paying for products and services that do not include any outcome guaranty or risk sharing.

The healthcare vendor 20 analyzes the plan of care 40 with respect to the population of the healthcare institution 30 to determine the types of equipment (e.g., support mattresses, powered mattresses, wheelchair cushions, geriatric chair cushions, recliner cushions, advanced therapy mattresses), consumables (e.g., skin maintenance products, wound care products), ancillary consultation services and materials (e.g., appropriate personnel for consultation, additional skin and wound care documentation materials and resources), and special products (e.g., alternative products and/or equipment necessary to ensure appropriate care of each resident 30 when the standard consumables are not sufficient to provide the necessary level of care) that are necessary to achieve the plan of care 40, both on a start-up and a continuing inventory control basis. This information is then utilized in the actuarial models to determine a fixed cost for the implementation of the management program 10.

In some embodiments, the healthcare vendor 20 sells the healthcare provider 15 a sufficient quantity of support mattresses to ensure that each customer's bed is equipped with a support mattress throughout the term of the management program 10. The cost of this equipment can be included in the fixed cost or invoiced separately. Utilization of new support mattresses provides stabilized costs for the healthcare provider 15 when the support mattresses are covered by warranties for the duration of the management program 10. The healthcare vendor 20 may similarly provide a supply of wheelchair, geriatric chair, recliner, and other appropriate types of therapy cushions.

The healthcare vendor 20 ensures that adequate levels of supply are available to the healthcare provider 15 at all times. Effective inventory control is utilized to minimize the number of emergency situations. If an emergency situation does arise, the healthcare provider 15 can contact the healthcare vendor 20 to determine the best plan of action to take. Although the management program 10 attains cost efficiencies based in part on the use of typically branded, but generically interchangeable products and standardized treatment protocols, some situations call for the use of other products and protocols. The healthcare vendor 20 generally must approve the use of such products or services in order for the costs associated with such products and services to be included in the fixed fee of the management program 10. Further, the management program 10 may cover the use of non-formulary products when deemed necessary by a physician providing care to an customer 25. If the healthcare provider 15 has any questions regarding an customer 25 that require further information, whether the questions are emergency or non-emergency, the healthcare provider 15 can contact the healthcare vendor 20 (e.g., via telephone, via email, by scheduling a chat through the website 200, or through an other means of communication).

When the employees of the healthcare institution 30 are trained and the documentation tools 45 of the management program 10 are initialized, the management program 10 is put into action. A compliance check-list is often completed to ensure all components of the management program 10 are in place. In one embodiment, the compliance check-list includes questions similar to the following: (1) are support mattresses in place, (2) have physical therapist/occupational therapist seating assessments completed, (3) are seating products in place, (4) are powered support surfaces fully implemented, (5) are formulary skin and wound care products in place, (6) is a connection for the website 200 established, (7) is necessary computer hardware installed and operational, (8) have employees of the healthcare institution 30 been trained, (9) is corporate standardization and approval of plan of care 40 completed, (10) has a compliance notification (i.e., notification describing the management program 10) been sent to all necessary employees, (11) have plans of care 40 been established and reviewed for each customer 25, (12) have referring physicians been informed of management program 10, and/or (13) has historical wound data been entered in website 200.

Care provided to customers 25 that have existing diseases at the beginning of the management program 10 is focused on reducing or eliminating the disease. Care provided to customers that do not have existing disease at the beginning of the management program 10 is focused on pressure management and skin maintenance. The plan of care 40 provides guidance on how to determine what type of care to provide an customer and what equipment and consumables to use to achieve that type of care. Once a disease is recognized, the applicable protocols are followed to determine how to best treat the disease. The disease may require upgraded pressure management devices, skin maintenance products, and/or wound care products.

Thus, the invention provides, among other things, a management program that provides healthcare providers with a comprehensive solution to a healthcare need. The management program is useful in reducing the risk of having adverse outcomes, and the costs of liability associated with those adverse outcomes, during the provision of healthcare.

Various features of the invention are set forth in the following claims.

What is claimed is:

1. A method of managing the provision of healthcare to at least one customer in a healthcare institution managed by a healthcare provider, the method comprising the acts of:
establishing a relationship between the healthcare provider and a healthcare vendor;
communicating a management program from the healthcare vendor to the healthcare provider, wherein the management program includes a plan of care, a documentation tool, and an indemnity instrument;
implementing the management program at the healthcare institution for a fixed fee; and
transferring a payment from the healthcare provider to the healthcare vendor for at least a portion of the fixed fee.

2. The method of claim 1, wherein the act of implementing the management program includes evaluating the healthcare provider and the healthcare institution to generate a baseline profile.

3. The method of claim 2, wherein the plan of care is generated based at least in part on the baseline profile.

4. The method of claim 1, wherein the plan of care includes a plurality of protocols.

5. The method of claim 4, wherein the plurality of protocols include skin care protocols.

6. The method of claim 4, wherein the plurality of protocols include wound care protocols.

7. The method of claim 4, wherein the plurality of protocols include pressure management protocols.

8. The method of claim 4, wherein the plurality of protocols include protocols developed using nationally accepted standards of care.

9. The method of claim 1, wherein the act of implementing the management program includes identifying a quantity of equipment and supplies necessary to achieve the plan of care.

10. The method of claim 9, wherein the equipment includes support surfaces.

11. The method of claim 9, wherein the supplies includes at least one of skin care products and wound care products.

12. The method of claim 1, wherein the act of implementing the management program includes providing equipment and supplies necessary to achieve the plan of care.

13. The method of claim 1, wherein the documentation tool include a website for entering and tracking data relating to the at least one customer.

14. The method of claim 13, wherein the website includes a wound page for displaying and entering information relating to a wound the at least one customer has.

15. The method of claim 13, wherein the website includes a specialist chat page for discussion information relating to the care of the at least one customer with a specialist.

16. The method of claim 15, wherein the specialist is a wound specialist.

17. The method of claim 13, wherein the website includes a facility page including information about the healthcare institution.

18. The method of claim 13, wherein the website includes a reporting tools page for generating reports for analyzing at least some of the data relating to the at least one customer.

19. The method of claim 1, wherein the indemnity instrument distributes financial risks associated with the provision of healthcare between the healthcare provider and the healthcare vendor.

20. The method of claim 1, wherein the indemnity instrument provides up to a set amount of money for each adverse outcome that results in a liability.

21. The method of claim 20, wherein the liability includes a settlement.

22. The method of claim 20, wherein the liability includes a judgement.

23. The method of claim 20, wherein the adverse outcome occurred while the healthcare provider was complying with the management program.

24. The method of claim 23, wherein the documentation tool is utilized to demonstrate such compliance.

25. The method of claim 1, wherein the indemnity instrument includes an agreement formed between the healthcare vendor and the healthcare provider.

26. The method of claim 1, wherein the indemnity instrument includes an agreement formed between the healthcare vendor, the healthcare provider, and an insurance company.

27. The method of claim 1, wherein the management program includes a skin and wound care management program.

28. The method of claim 1, wherein the fixed fee is based at least in part on a census of the healthcare institution.

29. The method of claim 1, wherein the management program provides a support surface for placement on a bed of each of the at least one customer.

30. A method of managing the provision of skin and wound care to at least one customer in a healthcare institution managed by a healthcare provider, the method comprising the acts of:
establishing a relationship between the healthcare provider and a healthcare vendor;
communicating a skin and wound care management program from the healthcare vendor to the healthcare provider, wherein the skin and wound care management program includes
a plan of care having a pressure management component, a skin maintenance component, and a wound care component,
a documentation tool for entering and tracking data relating to the at least one customer, and
an indemnity instrument which distribute financial risks associated with the provision of skin and wound care to the at least one customer between the healthcare provider and the healthcare vendor;
implementing the skin and wound care management program at the healthcare institution for a fixed fee; and
transferring a payment from the healthcare provider to the healthcare vendor for at least a portion of the fixed fee.

31. The method of claim 30, wherein the act of implementing the skin and wound care management program includes providing a selection of pressure management support surfaces.

32. The method of claim 30, wherein the act of implementing the skin and wound care management program includes providing a selection of pressure management seating products.

33. The method of claim 30, wherein the pressure management component reduces the incidence of pressure wounds and assists in the treatment of existing pressure wounds and wounds without pressure etiology.

34. The method of claim 30, wherein the act of implementing the skin and wound care management program includes providing at least one product selection tree for selecting a pressure management surface for the at least one customer.

35. The method of claim 30, wherein the act of implementing the skin and wound care management program includes providing an inventory of skin care products.

36. The method of claim 30, wherein the act of implementing the skin and wound care management program includes providing a skin care product usage guide.

37. The method of claim 30, wherein the act of implementing the skin and wound care management program includes providing an inventory of wound management materials.

38. The method of claim 30, wherein the act of implementing the skin and wound care management program includes providing a wound treatment protocol chart.
